(19) United States
(12) Patent Application Publication
(10) Pub. No.: US 2001/0046992 A1
(43) Pub. Date: Nov. 29, 2001

(54) Treatment of urinary tract infections with antibacterial oxazolidinones

(76) Inventor: Donald Herman Batts, Kalamazoo, MI (US)

Correspondence Address: Bruce Stein, Pharmacia & Upjohn Company, Global Intellectual Property, 301 Henrietta Street, Kalamazoo, MI, 49001, US

(21) Appl. No.: 09/809,447

(22) Filed: Mar. 15, 2001

Related U.S. Application Data
(60) Provisional application No. 60/190,961, filed on Mar. 22, 2000.

(51) Int. Cl. A61K 31/5377; A61K 31/496; A61K 31/422

(52) U.S. Cl. 514/235.5; 514/254.02; 514/374

(57) ABSTRACT

The present invention is a method of treating a warm blooded mammal who has a urinary tract infection caused by a Gram-positive organism who is in need of such treatment, which comprises administering to the mammal in need of such treatment a urinary therapeutically effective amount of an antibacterial oxazolidinone.

### CROSS-REFERENCE TO RELATED APPLICATIONS

This application claims the benefit of the following provisional application: U.S. Ser. No. 60/190,961, filed Mar. 22, 2000, under 35 USC 119(e)(i).

### BACKGROUND OF THE INVENTION

1. Field of the Invention

The present invention is the treatment of urinary tract infections (UTIs) with antibacterial oxazolidinones.

2. Description of the Related Art

Oxazolidinones are well known to those skilled in the art as gram positive anti-bacterial agents, see, for example, U.S. Pat. Nos. 5,688,792, 5,529,998, 5,547,950, 5,627,181, 5,700,799, 5,843,967, 5,792,765, 5,684,023, 5,861,413, 5,827,857, 5,869,659, 5,698,574, 5,968,962 and 5,981,528.

Urinary tract infections have been known and treated for many years. The usual pharmaceutical agents used to treat urinary tract infections include sulfa drugs, penicillins, cephalosporins, fluoroquinolones and tetracyclines. Although these antibiotics remain effective for many urinary tract infections, especially those due to Gram-positive bacteria have become increasingly resistant. Certain Gram-positive bacterial such as vancomycin resistant Enterococci, may be resistant to all antibiotics except for the antibacterial oxazolidinones.

### SUMMARY OF INVENTION

Disclosed is a method of treating a warm blooded mammal who has a urinary tract infection caused by a Gram-positive organism who is in need of such treatment which comprises administering to the mammal in need of such treatment a urinary therapeutically effective amount of an antibacterial oxazolidinone.

### DETAILED DESCRIPTION OF THE INVENTION

Oxazolidinones are a new class of gram positive antibacterial agents which are known to those skilled in the art, see for example U.S. Pat. No. 5,688,792. (S)-N-[[3-[3-fluoro-4-(4-morpholinyl)phenyl]-2-oxo-5-oxazolidinyl]methyl]acetamide, known as linezolid, the compound of Example 5 of U.S. Pat. No. 5,688,792 is known and has the following chemical structural formula:

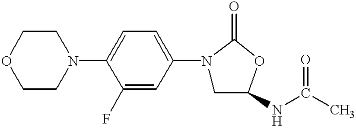

(S)-N-[[3-[3-fluoro-4-[4-(hydroxyacetyl)-1-piperazinyl]-phenyl]-2-oxo-5-oxazolidinyl]methyl]acetamide, known as eperezolid, the compound of Example 8 of U.S. Pat. No. 5,837,870 is known and has the following chemical structural formula:

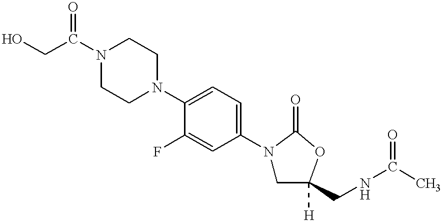

(S)-N-[[3-[3-fluoro-4-(tetrahydro-2H-thiopyran-4-yl)phenyl]-2-oxo-5-oxazolidinyl]methyl]acetamide S,S-dioxide the compound of Example 51of U.S. Pat. No. 5,968,962 has the following chemical structural formula:

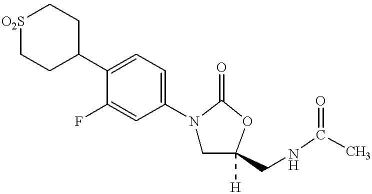

Linezolid and eperezolid can be produced by the processes set forth in U.S. Pat. Nos. 5,688,791 and 5,837,870 as well as that of International Publication WO99/24393. They are preferably produced by the process of U.S. Pat. No. 5,837,870. (S)-N-[[3-[3-fluoro-4-(tetrahydro-2H-thiopyran-4-yl)phenyl]-2-oxo-5-oxazolidinyl]methyl]acetamide S,S-dioxide can be produced by the process of U.S. Pat. No. 5,968,962 or the process of U.S. patent application Ser. No. 60/118,150; it is preferred that it be produced by the process of U.S. patent application Ser. No. 60/118,150.

It is preferred that linezolid produced be used in crystal form II, which has the characteristics set forth in CHART A. Once linezolid is synthesized, crystal Form II is prepared by starting with linezolid of high enantiomeric purity. It is preferred that the linezolid be more than 98% enantiomerically pure, it is more preferred that the linezolid be more than 99% pure and it is even more preferred that the linezolid be 99.5% pure. The linezolid of greater than 98% enantiomeric purity to be used to form crystal form II can either be in solution or be a solid. The linezolid starting material, solid or solution, is mixed with a solvent selected from the group consisting of compounds of the formula: water, acetonitrile, chloroform, methylene chloride, R₁—OH where R₁ is C₁-C₆ alkyl; R₁—CO-R₂ where R₂ is C₁-C₆ alkyl and R₁ is as defined above; phenyl substituted with 1 thru 3 R₁ where R₁ is as defined above; R₁—CO—O—R₂ where R₁ is C₁-C₆ alkyl and R₁ is as defined above; R₁—O—R₂ where R₁ is C₁-C₆ alkyl and R₁ is as defined above. It is preferred that the solvent be selected from the group consisting of water, ethyl acetate, methanol, ethanol, propanol, isopropanol, butanol, acetonitrile, acetone, methyl ethyl ketone, chloroform, methylene chloride, toluene, xylene, diethyl ether, or methyl-t-butyl ether. It is more preferred that the solvent be ethyl acetate, acetone, acetonitrile, propanol, or isopropanol. It is most preferred that the solvent be ethyl acetate. The mixture of linezolid in the solvent is agitated at a temperature below 80° until crystals of Form II are formed and crystals of other solid forms, such as Form I, disappear. It is preferred to dissolve the linezolid in ethyl acetate at a temperature near the boiling point of the solvent. This mixture is cooled to a temperature of about 70°. The mixture may be seeded with crystals of Form II to facilitate crystallization. It is preferred that the solid product is cooled and agitated at a temperature between about 45° and about 60° until the solids consist only of Form II crystals. It is most preferred to maintain the slurry at a temperature of about 55°. It is preferred to mix the linezolid and solvent for at least 10 min, it is even more preferred to mix the linezolid and solvent for at least 20 min and it is most preferred to mix the linezolid and solvent for at least 30 min. The time and temperature will vary depending on the solvent selected. With ethyl acetate it is preferred to mix for not less that 60 minutes. The crystalline slurry may be further cooled to improve yield, and the solid Form II product may be isolated. The mixture may be further cooled and agitated. Other measures which can be used to facilitate crystallization include, but are not limited to, cooling, concentration of the solution by evaporation or distillation, or through addition of other solvents. The crystals are isolated by procedures known to those skilled in the art.

To be useful pharmaceutical agents the antibacterial oxazolidinones must be compounded into pharmaceutical formulations. The preferred pharmaceutical formulations are an aqueous solution for IV administration, a tablet and a suspension for oral administration.

The preferred pharmaceutical formulation for IV administration, and the process to prepare it, are set forth in EXAMPLE 1.

The aqueous solution for IV administration can be placed in the container which is selected from the group consisting of a bag, a bottle, a vial, a large volume parenteral, a small volume parenteral, a prefilled syringe and a cassette. It is realized that a vial is a bottle. However, those skilled in the art use the term “bottle” to refers to larger bottles and “vials” to refer to smaller bottles. It is preferred that the container be a bag, a bottle, a vial or a prefilled syringe. It is more preferred that the container be a bag or bottle. It is most preferred that the container be a bag. The shape and/or size of the container is unimportant. It is preferred that the container be a bag sufficient to hold 25 to 2,000 mL of IV solution. It is preferred that the linezolid mixture be put in bags in amounts of 100, 200 or 300 mL of solution however smaller or larger volumes are acceptable.

It is well known to those skilled in the art that pharmaceutical agents administered IV must be sterile. While there are a number of methods to sterilize an IV solution, it is preferred to terminally moist heat or steam sterilize IV solutions of antibacterial oxazolidinones including those of linezolid. When the term terminally “moist heat sterilize” is used, it refers to and includes steam sterilization.

When terminally moist heat sterilizing an IV solution, the solution is placed in the container in which (1) it will be stored and then transferred to the container from which it will ultimately be administered, or (2) stored and then ultimately administered from the same container to deliver the IV solution to the patient. Therefore, it is imperative that the pharmaceutically active ingredient (oxazolidinone, linezolid) not react with the container in which it is to be terminally moist heat sterilized and stored/stored-administered.

It has been found that when the container-solution contact surface is made of at least 50% polyolefin there is significantly much less loss of linezolid during and following terminal moist heat sterilization. What is essential is that the container-solution contact surface material be primarily a polyolefin; the remainder of the container can be made from polyolefin or other materials. It is preferred that the container-solution contact surface is made of from about 50 to about 100% polyolefin. It is more preferred that the container-solution contact surface is made of from about 70 to about 90% polyolefin. It is more preferred that the container-solution contact surface is made of from about 80% polyolefin. It is even more preferred that the container-solution contact surface is made of polyolefin.

Polyolefins include, for example, polyethylene, polypropylene, polybutenes, polyisoprenes and polypentenes and copolymers and mixtures thereof. It is preferred that the polyolefin be selected from the group consisting of polyethylene and polypropylene. It is more preferred that the polyolefin be polypropylene or mixture of polypropylene and polyethylene.

The preferred tablet formulations and the process to prepare them are set forth in

### EXAMPLES 2 and 3 as well as CHART B.

The preferred suspension formulation is set forth in EXAMPLE 4.

Urinary tract infections occur in warm blooded mammals including humans, horses, cattle, dogs and cats. It is preferred that the mammal be a human. Adults as well as children get urinary tract infections as will be explained below.

Urinary tract infections are well known to those skilled in the art and are defined and the requirements for diagnosis are set forth in the textbook, Principals and Practice of Infectious Diseases, edited by Mandell, Bennett and Dolin, As is known to those skilled in the art urinary tract infections include infections of the kidney, bladder, urethra, ureter and asymptomatic bacteriuria. Technically, asymptomatic bacteriuria is not a urinary tract infection but may lead to one and is treated as such by those skilled in the art. It is preferred that the infection be of the bladder or of the kidney. Infections of the bladder are known as cystitis and those of the kidney as pyelonephritis. These infections may be caused by Gram-positive microorganisms as is well known to those skilled in the art. These Gram-positive microorganisms include for example Staphylococcus spp., Streptococcus spp., Enterococcus spp. Regardless of the particular microorganism the urinary tract infection is treated similarly, although Staphylococcus aureus infections may require longer courses of therapy. The major factor in deciding the specific treatment is the extent and severity of infection rather than the particular microorganism causing that infection. The presence of prosthetic material (e.g., urinary stents) or other complicating factors such as urinary stones or abscesses may also influence the dose, route of administration or duration of therapy.

It is preferred that the oxaxolidinone be selected from the group consisting of (S)-N-[[3-[3-fluoro-4-(4-morpholinyl)phenyl]-2-oxo-5-oxazolidinyl]methyl]acetamide, (S)-N-[[3-[3-fluoro-4-[4-(hydroxyacetyl)-1-piperazinyl]-phenyl]-2-oxo-5-oxazolidinyl]methyl]acetamide, (S)-N-[[3-[3-fluoro-4-(tetrahydro-2H-thiopyran-4-yl)phenyl]-2-oxo-5-oxazolidinyl]methyl]acetamide S,S-dioxide. It is more preferred that the antibacterial oxazolidinone be (S)-N-[[3-[3-fluoro-4-(4-morpholinyl)phenyl]-2-oxo-5-oxazolidinyl]methyl]acetamide.

The antibacterial oxazolidinone can be administered either IV or orally. The route of administration depends somewhat on the condition to be treated and the severity of the disease. For example, it is preferred to treat pyelonephritis IV while it is preferred to treat bladder infections orally. It is preferred oral administration be with a tablet or oral suspension. The antibacterial oxazolidinone is administered either two or three times daily depending on the place of the infection, the severity of the disease and the age of the mammal. For example young children less than or about 5 years of age clear the antibacterial oxazolidine very rapidly and may require three times a day administration. Also patients that do not respond well to twice daily dosing may require three times a day administration. All things being equal, twice daily administration is preferred.

The urinary therapeutically effective amount is from about 100 mg two times daily to about 600 mg three times daily. It is preferred that the urinary therapeutically effective amount is from about 200 mg twice daily to about 600 mg three times daily. It is more preferred that the urinary therapeutically effective amount is from about 400 mg to about 600 mg two times daily. For young children, especially those about age 5 and under, the preferred dose is about 10 mg/kg twice daily.

It is preferred that the human does not have a disease/condition selected from the group consisting of pneumonia; skin and soft tissue infections; does not have vancomycin resistant Enterococcus; arthritis; psoriasis or is not taking cancer chemotherapy.

The exact dosage and frequency of administration depends on the particular antibacterial oxazolidinone used, the particular urinary tract infection being treated, the severity of the condition being treated, the age, weight, general physical condition of the particular patient, other medication the individual may be taking as is well known to those skilled in the art and can be more accurately determined by measuring the blood level or concentration of the antibacterial oxazolidinone in the patient's blood and/or the patient's response to the particular condition being treated.

### DEFINITIONS AND CONVENTIONS

The definitions and explanations below are for the terms as used throughout this entire document including both the specification and the claims.

### DEFINITIONS

All temperatures are in degrees Celsius.

IV refers to intravenous.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When the solubility of a solid in a solvent is used the ratio of the solid to the solvent is weight/volume (wt/v).

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

Linezolid IV Solution

The composition of Linezolid IV solution is as follows:

| | | |
|---|---|---|
| Linezolid | 2.0 | mg |
| Dextrose, USP | 50.24 | mg |
| Sodium citrate, USP | 1.64 | mg |
| Citric acid, USP | 0.85 | mg |
| Water for injection, USP | q.s. ad 1 | ml |

The linezolid IV solution is formulated by heating water for injection to 60°. Next the sodium citrate, citric acid and dextrose are added and stirred until dissolved. An aqueous slurry of linezolid is added to the previous mixture and stirred until dissolved. The mixture is cooled to 25° with stirring. The pH is measured and adjusted if necessary. Last the mixture is brought to volume, if necessary with water for injection. The mixture is filtered, filled into infusion containers, over wrapped and terminally moist heat sterilized.

### Example 2

Linezolid (400 mg) Tablet Formulation Ingredients Amount

| Ingredients | Amount | |
|---|---|---|
| Linezolid | 400.0 | mg |
| Corn starch NF | 40.0 | mg |
| Microcrystalline cellulose NF | 16.0 | mg |
| Hydroxypropylcellulose (intragranular) NF | 5.92 | mg |
| Hydroxypropylcellulose (binder solution) NF | 2.08 | mg |
| Microcrystalline cellulose NF | 62.4 | mg |
| Sodium starch glycolate NF | 28.0 | mg |
| Magnesium stearate NF | 5.6 | mg |
| Purified water USP 22.0% uncoated tablet wt | | |
| Film Coating Phase | | |
| Opadry White Y-1-18202-A | 16.8 | mg |
| Purified Water USP | 129.2 | mg |
| Polishing Phase | | |
| Carnauba Wax NF | 0.0224 | mg |

The binder solution is prepared by adding part of the hydroxypropylcellulose to the purified water and mixing in an appropriate container until dissolved. The granulation is performed by adding the antibacterial oxazolidinone, corn starch, microcrystalline cellulose (intragranular, 24.0 mg), and the remaining hydroxypropylcellulose into a high shear mixer and mixing until adequately mixed. Then add the binder solution while mixing, and if needed, add an additional sufficient quantity of water while mixing, to form the granulation. Wet screen the granulation using appropriate equipment as is well known to those skilled in the art, for example, a Comil. Following granulation the granulation is dried using suitable equipment, such as a fluid bed dryer. After the granulation is dried, dry screen the granulation using appropriate equipment, such as a Comil. The lubrication portion is formed by mixing microcrystalline cellulose (extragranular, 93.6 mg) and sodium starch glycolate with the dry screened granulation in a suitable blender, such as a diffusion (tumble) type V blender, until adequately blended. Next remove a portion of the blended material and combine with the magnesium stearate. Add the magnesium stearate mixture back into the blender, such as a diffusion (tumble) type V-blender, and mix until adequately blended. Finally, collect the lubricated powder mixture in appropriate containers.

The compressed tablets are formed using a suitable rotary compression machine and compression tooling. The lubricated powder mixture is compressed into tablets of proper weight, hardness, size and shape.

It is preferred to coat and wax the tablets. The Opadry White Y-1-18202-A and purified water are mixed to prepare the coating suspension. The coating mixture should be continuously stirred until the mixture is free of lumps and the Opadry is in suspension. Prior to using, the film coating suspension should be screened through an appropriate screen. The desired quantity of tablets is loaded into the appropriately sized perforated coating pans (such as an Accela-Cota or Glatt coating pan) equipped with baffles, spray guns and pumping system. The appropriate amount of aqueous film coating is sprayed on the moving tablets until tablets are evenly coated. After the coating is complete, the appropriate amount of carnauba wax is weighed and added to the bed of tablets to polish the film coated tablets.

It is preferred to print the tablets with identifying information as is known to those skilled in the art.

A flow chart of the manufacturing process is provided in CHART B.

### Example 3

Linezolid (600 mg) Tablet Formulation Ingredients Amount

| Ingredients | Amount | |
|---|---|---|
| Linezolid | 600.0 | mg |
| Corn starch NF | 60.0 | mg |
| Microcrystalline cellulose NF | 24.0 | mg |
| Hydroxypropylcellulose (intragranular) NF | 8.88 | mg |
| Hydroxypropylcellulose (binder solution) NF | 3.12 | mg |
| Microcrystalline cellulose NF | 93.6 | mg |
| Sodium starch glycolate NF | 42.0 | mg |
| Magnesium stearate NF | 8.4 | mg |
| Purified water USP 22.0% uncoated tablet wt | | |
| Film Coating Phase | | |
| Opadry White Y-1-18202-A | 25.2 | mg |
| Purified Water USP | 193.9 | mg |
| Polishing Phase | | |
| Camauba Wax NF | 0.0336 | mg |

Following the general procedure of EXAMPLE 1 and making non-critical variations but using the amounts of ingredients above Linezolid 600 mg tablets are produced.

### Example 4

Suspension Formulation

| | | |
|---|---|---|
| Linezolid | 100.0 | mg |
| Xanthan Gum NF Food Grade | 15.0 | mg |
| Avicel RC-591 | 50.0 | mg |
| Sucrose NF Granular | 1,052.9 | mg |
| Mannitol USP powder | 500.0 | mg |
| Sodium citrate USP hydrous | 15.0 | mg |
| Citric acid USP anhydrous | 9.1 | mg |
| Aspartamine NF powder | 35.0 | mg |
| Collodial silicon dioxide NF | 15.0 | mg |
| Sodium Benzoate | 10.0 | mg |
| Sodium Chloride | 13.5 | mg |
| Sweet-AM-Powder | 30.0 | mg |
| Mafco Magnasweet #135 | 60.0 | mg |
| NOR-CAP Orange Flavor | 50.0 | mg |
| NOR-CAP Orange Cream Flavor | 37.5 | mg |
| Pepperemint Flavor | 2.0 | mg |
| Vanilla flavor | 5.0 | mg |

The suspension is prepared by methods well known to those skilled in the art.

### Chart A

Linezolid, (S)-N-[[3-[3-fluoro-4-(4-morpholinyl)phenyl]-2-oxo-5-oxazolidinyl]methyl]acetamide, crystal “Form II” has the powder X-ray diffraction spectrum of:

| d-Spacing (Á) | Two-Theta Angle (°) | Relative Intensity (%) |
|---|---|---|
| 12.44 | 7.10 | 2 |
| 9.26 | 9.54 | 9 |
| 6.37 | 13.88 | 6 |
| 6.22 | 14.23 | 24 |
| 5.48 | 16.18 | 3 |
| 5.28 | 16.79 | 100 |
| 5.01 | 17.69 | 2 |
| 4.57 | 19.41 | 4 |
| 4.50 | 19.69 | 2 |
| 4.45 | 19.93 | 6 |
| 4.11 | 21.61 | 15 |
| 3.97 | 22.39 | 23 |
| 3.89 | 22.84 | 4 |
| 3.78 | 23.52 | 7 |
| 3.68 | 24.16 | 1 |
| 3.52 | 25.28 | 13 |
| 3.34 | 26.66 | 1 |
| 3.30 | 27.01 | 3 |
| 3.21 | 27.77 | 1 |

and an infrared (IR) spectrum (mineral oil mull) of 3364, 1748, 1675, 1537, 1517, 1445, 1410, 1401, 1358, 1329, 1287, 1274, 1253, 1237, 1221, 1145, 1130, 1123, 1116, 1078, 1066, 1049, 907, 852 and 758 cm⁻¹.

### Chart B

### Linezolid Tablet Manufacturing Flowchart

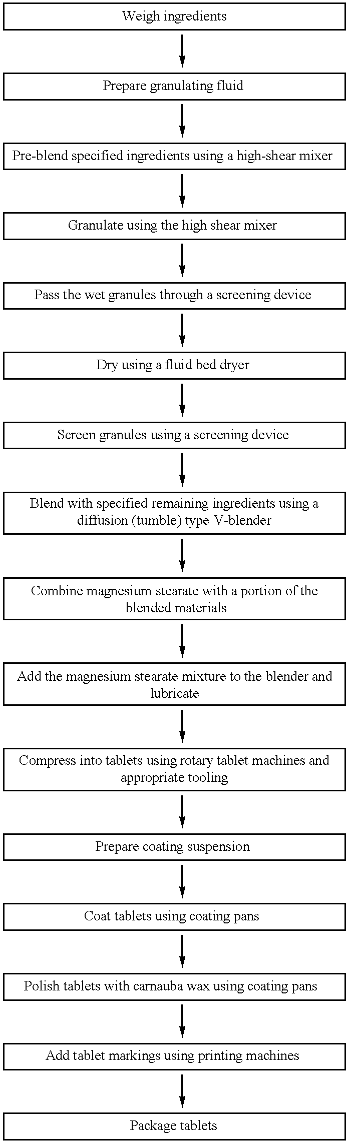

1. A method of treating a warm blooded mammal who has a urinary tract infection caused by a Gram-positive organism who is in need of such treatment which comprises administering to the mammal in need of such treatment a urinary therapeutically effective amount of an antibacterial oxazolidinone.

2. A method of treating a warm blooded mammal according to claim 1 where the mammal is a human.

3. A method of treating a warm blooded mammal according to claim 1 where the urinary tract infection is an infection is selected from the group consisting of infections of the kidney, bladder, urethra, ureter and asymptomatic bacteriuria.

4. A method of treating a warm blooded mammal according to claim 3 where the urinary tract infection is an infection of the bladder.

5. A method of treating a warm blooded mammal according to claim 3 where the urinary tract infection is an infection of the kidney.

6. A method of treating a warm blooded mammal according to claim 1 where the antibacterial oxazolidinone is selected from the group consisting of
(S)-N-[[3-[3-fluoro-4-(4-morpholinyl)phenyl]-2-oxo-5-oxazolidinyl]methyl]acetamide,
(S)-N-[[3-[3-fluoro-4-[4-(hydroxyacetyl)-1-piperazinyl]-phenyl]-2-oxo-5-oxazolidinyl]methyl]acetamide,
(S)-N-[[3-[3-fluoro-4-(tetrahydro-2H-thiopyran-4-yl)phenyl]-2-oxo-5-oxazolidinyl]methyl]acetamide S,S-dioxide.

7. A method of treating a warm blooded mammal according to claim 6 where the antibacterial oxazolidinone is (S)-N-[[3-[3-fluoro-4-(4-morpholinyl)phenyl]-2-oxo-5-oxazolidinyl]methyl]acetamide.

8. A method of treating a warm blooded mammal according to claim 6 where the antibacterial oxazolidinone (S)-N-[[3-[3-fluoro-4-[4-(hydroxyacetyl)-1-piperazinyl]-phenyl]-2-oxo-5-oxazolidinyl]methyl]acetamide.

9. A method of treating a warm blooded mammal according to claim 6 where the antibacterial oxazolidinone is (S)-N-[[3-[3-fluoro-4-(tetrahydro-2H-thiopyran-4-yl)phenyl]-2-oxo-5-oxazolidinyl]methyl]acetamide S,S-dioxide.

10. A method of treating a warm blooded mammal according to claim 1 where the antibacterial oxazolidinone is administered orally.

11. A method of treating a warm blooded mammal according to claim 1 where the antibacterial oxazolidinone is administered IV.

12. A method of treating a warm blooded mammal according to claim 1 where the antibacterial oxazolidinone is administered 2 or 3 times daily.

13. A method of treating a warm blooded mammal according to claim 12 where the antibacterial oxazolidinone is administered 2 times daily.

14. A method of treating a warm blooded mammal according to claim 1 where the urinary therapeutically effective amount is from about 100 mg two times daily to about 600 mg three times daily.

15. A method of treating a warm blooded mammal according to claim 14 where the urinary therapeutically effective amount is from about 200 mg twice daily to about 600 mg three times daily.

16. A method of treating a warm blooded mammal according to claim 15 where the urinary therapeutically effective amount is from about 400 mg to about 600 mg two times daily.

17. A method of treating a warm blooded mammal according to claim 1 where the human does not have a disease or condition selected from the group consisting of pneumonia; skin and soft tissue infections; does not have vancomycin resistant Enterococcus; arthritis; psoriasis or is not taking cancer chemotherapy.

18. A method of treating a warm blooded mammal according to claim 17 where the human does not have pneumonia.

19. A method of treating a warm blooded mammal according to claim 17 where the human does not have a skin and soft tissue infections.

20. A method of treating a warm blooded mammal according to claim 17 where the human does not have a vancomycin resistant Enterococcus infection.

21. A method of treating a warm blooded mammal according to claim 17 where the human does not have arthritis.

22. A method of treating a warm blooded mammal according to claim 17 where the human does not have psoriasis.

23. A method of treating a warm blooded mammal according to claim 17 where the human is not taking cancer chemotherapy.
